(19) United States
(12) Patent Application Publication
(10) Pub. No.: US 2005/0074901 A1
(43) Pub. Date: Apr. 7, 2005

(54) Methods and assays for detecting and quantifying the human p53 inducible gene protein

(76) Inventors: John Castracane, Groton, MA (US); Alison Joyce, Groveland, MA (US); Thomas Miller, East Boston, MA (US); Fred H. Reynolds, Waltham, MA (US)

Correspondence Address: MIRICK O'CONNEL;MIRICK, O'CONNELL, DEMALLIE & LOUGEE, LLP, 1700 West Park Drive, WESTBOROUGH, MA, 01581-3941, US

(21) Appl. No.: 10/367,505

(22) Filed: Feb. 13, 2003

Related U.S. Application Data
(60) Provisional application No. 60/356,287, filed on Feb. 13, 2002.

(51) Int. Cl. G01N 33/543

(52) U.S. Cl. 436/518

(57) ABSTRACT

Methods, assays, and kits for detecting the presence of PIG3 in human cell extracts, serum, plasma, other body fluids and extra-cellular tissue culture media to determine valuable prognostic, monitoring and diagnostic information about the status of a patient. The methods are rapid and quantitative and can be used to evaluate the viability of cells and tissue, to evaluate progress of a disease and/or its treatment, to evaluate the cytotoxicity of unknown compounds and to study the kinetics of cell death.

### CROSS REFERENCE TO RELATED APPLICATION

This application claims priority of Provisional application Ser. No. 60/356,287, filed on Feb. 13, 2002.

### FIELD OF THE INVENTION

The invention features novel methods and assays for detecting and quantifying the human p53 inducible gene (PIG3) protein in human cell extracts, serum, plasma, other body fluids and extra-cellular tissue culture media.

### BACKGROUND AND SUMMARY OF THE INVENTION

The methods of the invention for detecting and quantifying the human p53 inducible gene (PIG3) protein in human cell extracts, serum, plasma, other body fluids and extra-cellular tissue culture media provide invaluable information not previously available. For example, detecting and quantifying the human p53 inducible gene (PIG3) protein levels in the serum, plasma, and urine of chemotherapy patients using the methods and immunoassays of the invention provide valuable prognostic, monitoring and diagnostic information about the status of a patient undergoing chemotherapy. The methods also enable one to determine risk-assessment of suspected genotoxins, evaluation of novel chemotherapeutic agents, and novel chemotherapeutic methods for cancer management. The immunoassay of the invention provides information about tolerance to chemotherapeutic agents, toxicity and/or side effects that can result in discontinuation of therapy, patient compliance with therapy due to side effects, and improved modality of administration (dosimetry) of chemotherapy agents to maximize efficacy and improve dosing to prevent events such as the induction of DNA repair systems in the case of chemotherapy agents that directly (cisplatin and platin family of drugs) or indirectly effect or damage DNA and induce or otherwise result in the expression of the p53 inducible gene 3. Administration of other agents may similarly be used to monitor DNA replication (cisplatin), microtubules (Taxol), growth signaling pathways (Gleevec) or compounds that work in blocking the cell's proteasome activity or function. It is envisioned that other p53 inducible genes (more than 20 in all) may alternatively be used with the methods of the invention to the extent they are functionally comparable to PIG3. PIG3 measurements derived using the methods and assays of the invention are also intended to screen for carcinogenic (environmental exposure) agents.

It now has been discovered that the cytoplasmic protein PIG3, or soluble fragments thereof, are released in soluble form from cells undergoing p53 induced cell death (and perhaps as a result of other mechanisms not yet identified as in the taxol induction of PIG3). As used herein, “cell death” is understood to include both apoptosis and “programmed” cell death, but not necrosis. Moreover, it also now has been discovered that, using the assays and methods of the invention, these soluble forms of the cytoplasmic protein PIG3 can be quantitated in a fluid and used to monitor the degree, or rate, of cell death in a tissue. The methods and assays of the invention also may be used to distinguish between types of cell death occurring. The presence of this protein and protein fragments in solution in soluble form in a body fluid is unexpected as this molecule is a cytoplasmic protein believed to be involved in the apoptotic process within a cell under standard physiological conditions. The discovery of this protein released in soluble form from dying cells allows one to use the soluble cytoplasmic protein PIG3 as biochemical monitor of p53-specific cell death in fluid assays. The methods are rapid and quantitative and can be used to evaluate the viability of cells and tissue, to evaluate progress of a disease and/or its treatment, to evaluate the cytotoxicity of unknown compounds and to study the kinetics of cell death. The discovery also provides an alternative method for purifying the PIG3 protein from cells.

It is a primary object of this invention to provide methods and assays for monitoring the degree of p53 mediated cell death in a body fluid assay by monitoring the level of body fluid-soluble PIG3 a fluid.

It is a further object of the invention is to provide methods and assays for quantifying the body fluid-soluble PIG3 released from cells and present in a cell compatible fluid.

It is a further object of the invention is to provide methods and assays for evaluating a therapy, or the progress of a disease associated with p53 mediated cell death by monitoring the degree of cell death in an affected tissue.

It is a further object of the invention is to provide methods and assays for assessing the toxicity of compounds by assessing their ability to induce p53 and p53 mediated cell death.

It is a further object of the invention is to provide methods for inducing the release of body fluid-soluble PIG3 proteins from eucaryotic cells.

It is a further object of this invention to provide methods and assays for monitoring and dosimetry of (correct dosage determination of) chemotherapeutic agents through the measurement of PIG3 protein in human sera and plasma samples, tissue extracts (normal and tumor tissue) and cell culture supernatants. This may be done with the measurement of PIG3 alone or in conjunction with DNA adduct level determination or other measurement of DNA damage induced by chemotherapeutic or genotoxic agents.

It is a further object of the invention to provide methods and assays for the in vitro measurement of PIG3 in tissue culture samples obtained from patients before the onset of chemotherapy treatment where toxicity, and efficacy may be determined prior to treatment with these agents in order to positively affect the outcome of treatment, the tolerance to chemotherapeutic or genotoxic agents and avoid the administration of increasing amounts of agent with time (which is currently practiced in order to determine the maximum tolerable dose on an individual patient basis) the administration of chemotherapeutic or genotoxic agents in increasing doses with time serves to induce tolerance to these agents in patients thereby negating any therapeutic benefit to the patients.

It is a further object of the invention to determine the maximum tolerable dose that may be administered to avoid induction of tolerance or DNA repair systems in the patient while determining levels of chemotherapeutic or genotoxic agents that induce the minimum toxicity needed to affect treatment thereby increasing patient compliance and quality of life during treatment.

The methods and assays of the invention generally involve detecting the concentration of body fluid-soluble cytoplasmic protein PIG3 or fragments thereof released from cells and comparing this concentration to a known standard. The concentration of these proteins in a body fluid sample is indicative of the degree of cell death in that tissue. Fluid samples are collected at discrete intervals and detected by means, for example, of an immunoassay. The concentrations of PIG3 then are compared, with changes in concentrations being indicative of the changes in the rate of cell death. Exemplary body fluids include blood, serum, plasma, urine, semen, spinal fluid, saliva, ascites fluid, peritoneal fluid, sputum, tissue swabs, and body exudates such as breast. Alternately, cells may be collected and extracts prepared and measured for total PIG3 protein. An example of cells that may be collected is peripheral blood lymphocytes, tissue biopsy samples, cells collected from the mouth by scrapping, swabs or other techniques commonly in practice. Both normal and tumor tissue may be assessed in this assay. The determination of PIG3 induction in (or released by) a representative normal tissue indicative of toxicity and the measurement of PIG3 in (or released by) a tumor tissue in response to in vivo exposure, in vitro exposure or ex vivo exposure to a chemotherapy agent or combinations of chemotherapy agents is indicative of the effectiveness of the treatment.

The methods may be used to evaluate the progress of a disease. For example, the methods may be used to monitor the progress of a malignancy such as, for example, a carcinoma, adenoma, sarcoma, lymphoma, or myeloma. Here quantitating the level of soluble PIG3 released from the malignant cells monitors the rate of cell death in a malignant tissue. Alternatively, the method also is used to monitor the progress of tissue disorders resulting in altered cell death, such as results from tissue atrophy, hyperplasia, cirrhosis, hypoxia, ischaemia and benign tumor growths. Injured tissue also can be assessed by this method, including direct cell trauma such as from membrane-active chemicals and toxins or resulting from direct physical trauma, such as, for example, hyperthermia, hypoxia and ischemia/reperfusion, radiation or compliment-mediated autolysis.

The methods may also be used to monitor the efficacy of a therapy. Here a therapeutic agent or procedure (e.g., radiation therapy) is administered to a patient and, thereafter, the concentration of soluble PIG3 or fragments are detected in body fluid samples drawn from the patient at predetermined intervals. These concentrations then are compared to each other and to those in samples tested before administration of the therapy. The changes in concentration of soluble PIG3 protein detected among the samples compared will be indicative of the therapy's efficacy. For example, a therapeutic agent capable of selectively destroying malignant cells can cause an increase in soluble PIG3 protein released from the tumor cells, followed by a reduction in the level of this protein detected as the number of malignant cells fall.

The methods are also used to monitor the effect of a given therapy on the viability of normal, unaffected tissue. For example, a number of cancer therapies, including radiation and chemotherapy, target rapidly proliferating cell populations and therefore can affect normally proliferative cell populations such as bone marrow progenitor cell populations and intestinal epithelial cells. The effect of the therapy on these cell populations can be evaluated by monitoring the level of soluble PIG3 protein released from these cells. Similarly, the method also is used to evaluate the efficacy of complimentary therapeutic agents designed to protect normal tissue from the effects of the therapy.

The methods are further advantageously used to monitor the toxic effects of a given therapy on the viability of normal, unaffected tissue. For example, a number of cancer therapies, including radiation and chemotherapy, target rapidly proliferating cell populations and therefore can affect normally proliferative cell populations such as bone marrow progenitor cell populations and intestinal epithelial cells. Such a use allows for the proper dosing of therapeutic agent to maximize tumoricial effects and minimize non-specific toxicities while increasing patient compliance with treatment and increase quality of life for those receiving treatments.

Still further, the methods are used to evaluate the type of cell death occurring and to study its kinetics. For example, PIG3 protein or protein fragments are identified that are released in soluble form by cells undergoing apoptosis versus necrosis, since it is believed that the PIG3 is one of the proteins encoded by p53 activated genes that modulate the production of reactive oxygen species (ROS), the increased production of ROS in turn damage mitochondria. Leakage of calcium and proteinaceous components from damaged mitochondria then stimulates the caspases that are activated during the apoptotic process. Since by definition this pathway does not occur during necrosis, PIG3 is present only at low concentrations in necrotic compared to p53 induced apoptotic cells. The rate of release of these proteins or protein fragments then may be monitored to investigate cell death kinetics.

Still further, the methods may be used to monitor the status of a cell culture and/or to assess the cytotoxicity of a compound by monitoring the levels of soluble the cytoplasmic protein PIG3 or protein fragments released from these cells. The methods are also used to induce release of body fluid-soluble cytoplasmic protein PIG3 from intact cells, using compounds capable of inducing apoptosis. The methods of the invention are useful as part of a protocol to identify candidate compounds useful as cancer chemotheapeutic agents.

A preferred method of the invention for detecting the presence of PIG3 in a specimen, generally comprises the steps of: (a) contacting a specimen with a solid support having immobilized thereon a first binding partner that binds specifically to a first binding site on a specimen-soluble PIG3 under conditions that permit said PIG3, if present in said specimen, to bind specifically to said first binding partner; (b) contacting said solid support produced in step (a) with a second binding partner that binds specifically to a second binding site on said PIG3 under conditions that permit said second binding partner to bind specifically to said PIG3 thereby to produce an immobilized first binding partner-PIG3-second binding partner complex; (c) contacting said complex with a third binding partner labeled with a detectable moiety, wherein said third binding partner binds specifically to said second binding partner, under conditions that permit said third binding partner to bind specifically to said second binding partner of said complex; (d) measuring the amount of detectable moiety immobilized to said solid support, wherein the amount of immobilized detectable moiety is indicative of the quantity per unit volume of said PIG3 in said sample, thereby to determine the quantity per unit volume of said PIG3 in said specimen; and (e) comparing the quantity per unit volume of said PIG3 with a preselected value indicative of the presence of said degree of p53 activation and induction of cell death, wherein a quantity of said PIG3 in said sample greater than said preselected value is indicative of the presence of said degree of p53 activation and induction of cell death in said specimen. The specimen may comprise one or more body fluids selected from a group consisting of, blood, serum, plasma, urine, semen, spinal fluid, ascitic fluid, peritoneal fluid, saliva, sputum, and breast exudate. Further, the degree of p53 activation and induction of cell death may be caused by a disease condition, treatment of a disease, or progress of a disease, wherein the disease may be a form of cancer including, but not limited to, carcinoma, adenoma, sarcoma, lymphoma or myeloma, bladder cancer, colon cancer, lung cancer, ovarian cancer, breast cancer, cervical cancer, kidney cancer, liver cancer, or uterine cancer.

The first and second binding partners are preferably antibodies, wherein the second binding partner is preferably a biotinylated antibody. The third binding partner is preferably streptavidin. The measurement of PIG3 may be particularly indicative of the efficiency of cell killing by radiation or chemotherapy.

A preferred method of the invention for determining the optimum dosimetry of a therapeutic agent for a patient, generally comprises the steps of: administering a first dose of said therapeutic agent to said patient; measuring one or more quantity per unit volume of PIG3 in one or more specimens taken from said patient; comparing said quantity per unit volume of said PIG3 with a preselected value that is indicative of cell death induction; and administering a second dose of said therapeutic agent to said patient based on said step of comparing said quantity per unit volume of said PIG3 with said preselected value that is indicative of cell death induction.

A preferred test kit of the invention, that is useful for performing an immunoassay to detect the presence of PIG3 in a specimen, generally comprises one or more monoclonal antibodies specific for the human PIG3 protein, wherein one or more of said monoclonal antibodies is monoclonal antibody X1155P and/or X1156M. Also, one or more of said monoclonal antibodies is preferably a biotinylated detector monoclonal antibodym, however, the test kit may comprise one or more immunologically responsive substances selected from a group consisting of horseradish peroxidase-conjugated streptavidin; radioactive agents, chemiluminescence agents, bioluminescence agents, fluorescence agents, or other chromophoric agents. One or more of said immunologically responsive substances preferably comprises horseradish peroxidase-conjugated streptavidin to catalyze conversion of a chromogenic substrate tetra-methylbenzidine. One or more of said monoclonal antibodies may be directly or indirectly labeled.

### BRIEF DESCRIPTION OF THE FIGURES

Other objects, features and advantages will occur to those skilled in the art from the following description of the preferred methods and assays of the invention and the accompanying figures in which:

FIG. 1 shows levels of PIG3 detected after extraction with a PIG3 antibody;

FIG. 2 shows dilution-recovery of positive samples of cell lysates for PIG3, sera, and plasma for soluble PIG3;

FIG. 3A shows the mean signal of each standard run in replicates of three in eight assays using two different of plates, detector antibody, and standards;

FIG. 3B illustrates the lower limit of detection measured by assaying replicates of zero four times using different lots of plates and detector antibody;

FIG. 3C is a precision profile showing pooled coefficients of variation and between assay coefficients of variation plotted against PIG3 levels;

FIG. 4A shows Adriamycin upregulation of PIG3 over time and dose;

FIG. 4B shows level of PIG3 in tissue culture supernatants of drug-treated cells over time;

FIG. 4C shows level of PIG3 in tissue culture supernatants of Cisplatin-treated cells over time; and

FIG. 5 shows drug induced in vitro PIG3 levels for cancer patient sera and normal human sera.

### DESCRIPTION OF THE PREFERRED METHODS AND ASSAYS

The invention features a PIG3 ELISA that is a “sandwich” enzyme immunoassay employing two mouse monoclonal antibodies and methods for using the assay. A monoclonal antibody, specific for the human PIG3 protein, is immobilized onto the surface of microtiter wells provided. A kit for the immunoassay, comprising two monoclonal antibodies X1155P and X1156M, is available from Exalpha Biologicals, Inc. in Boston, Mass. As the Frontier Assay Kit. The sample to be assayed and the biotinylated detector monoclonal antibody are pipetted into the wells and allowed to incubate for two hours, during which time any PIG3 present binds to the capture and detecting antibodies. Unbound material is washed away and horseradish peroxidase-conjugated streptavidin is added, which binds to the detector antibody.

The horseradish peroxidase catalyzes the conversion of the chromogenic substrate tetra-methylbenzidine (TMB) from a colorless solution to a blue solution (or yellow after the addition of stop reagent), the intensity of which is proportional to the amount of PIG3 protein in the sample. The colored reaction product is quantified using a spectrophotometer.

Alternatively, other comparable agents may be used in place of the streptavidin-biotin-horseradish peroxidase labeled immunologically responsive substance, such as radioactive agents, chemiluminescence agents, bioluminescence agents, fluorescence agents, or other chromophoric agents. In addition the detector antibody can be directly labeled or labeled indirectly with other molecules in place of biotin.

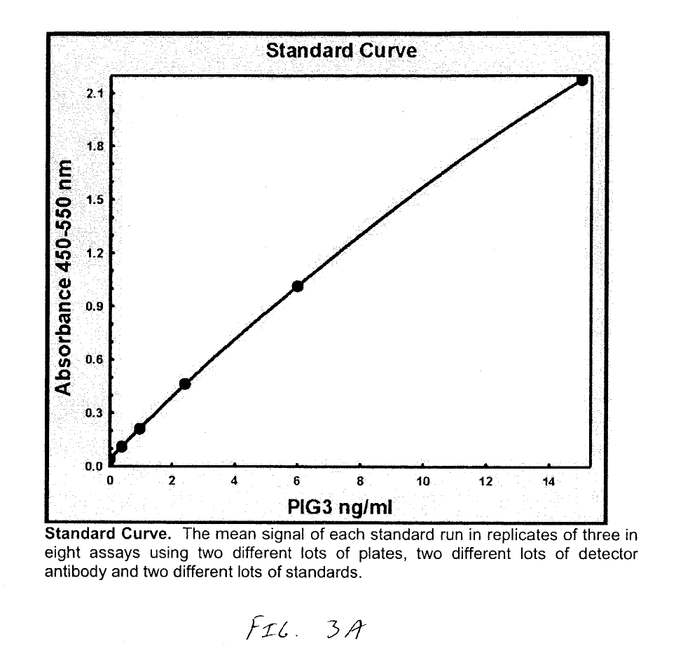

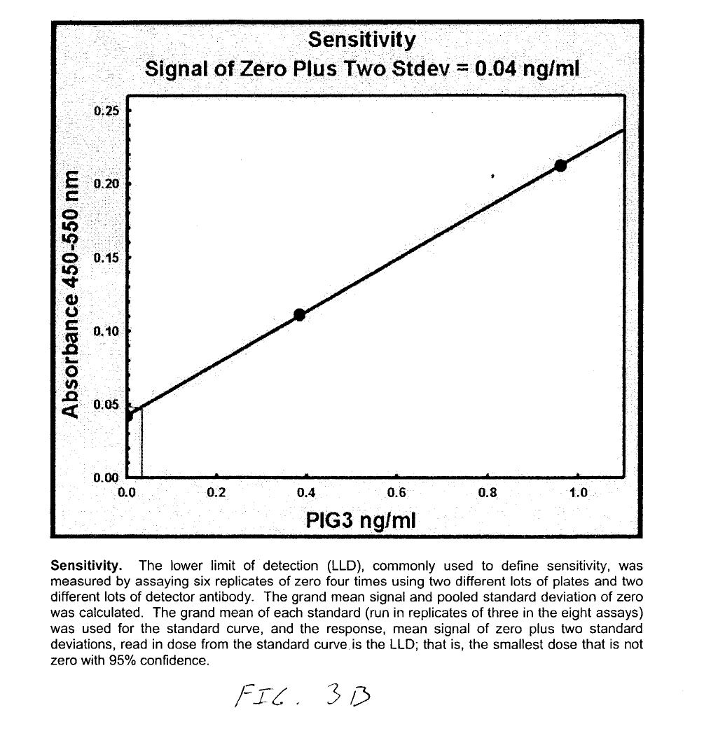

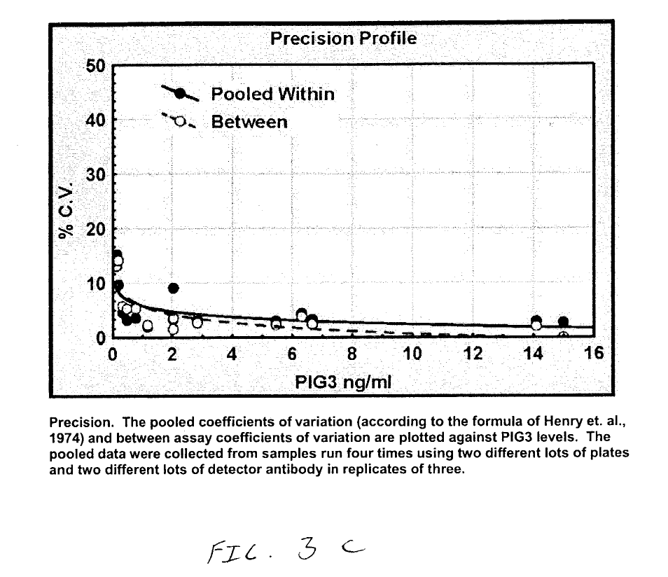

Quantitation is achieved by the construction of a standard curve using known concentrations of PIG3 (provided lyophilized). By comparing the absorbance obtained from a sample containing an unknown amount of PIG3 with that obtained from the standards, the concentration of PIG3 in the sample is determined. (FIGS. 3A-3C)

The subject invention utilizes innovative combinations of experimental techniques to provide commercially useful methods not previously taught. Several key innovative steps for detecting and quantifying soluble human PIG3 protein are utilized.

The invention generally features novel assays and methods for determining the degree of p53 activation and induction of cell death in a tissue by detecting and quantitating a soluble or otherwise released form of a cytoplasmic protein known as PIG3, the product of p53 inducible gene 3 in body fluids, tissue extracts and extracellular media. The methods are useful for monitoring the viability of cells and tissue, for evaluating the progress of a disease and/or its treatment, for determining the optimum dosimetry for therapeutic agents, and for evaluating the cytotoxicity of unknown compounds.

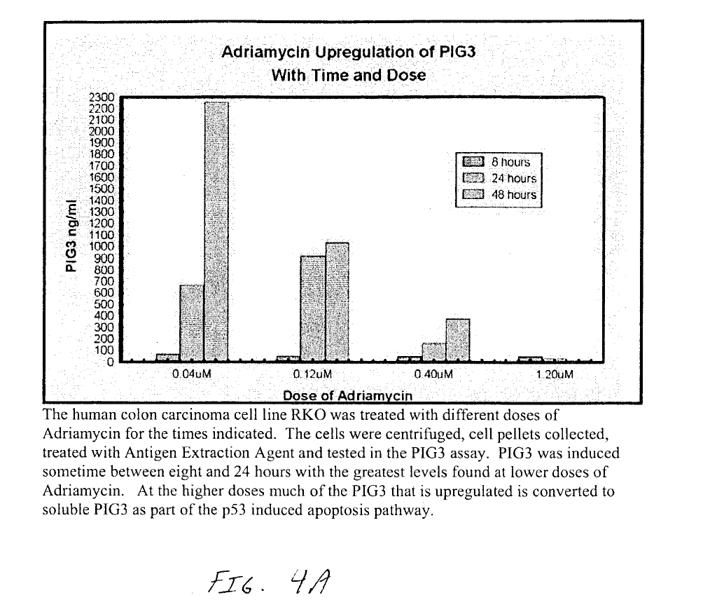

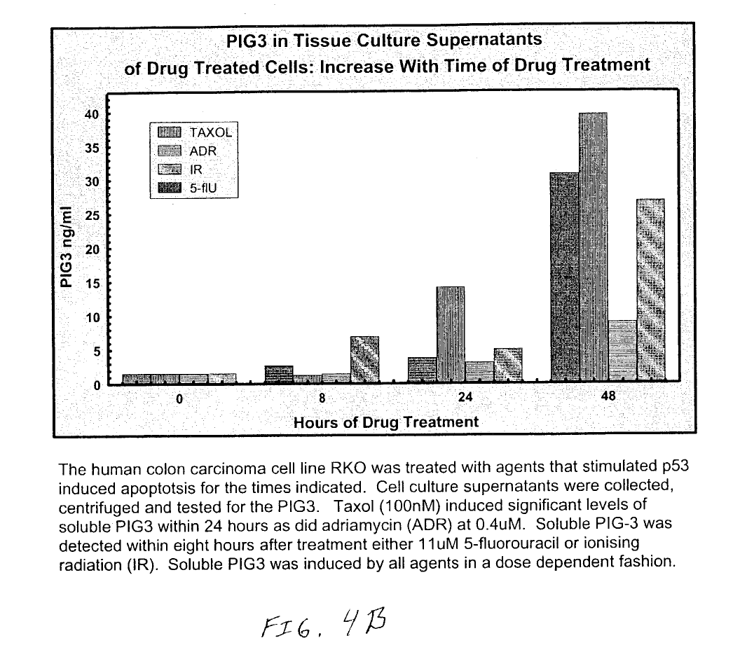

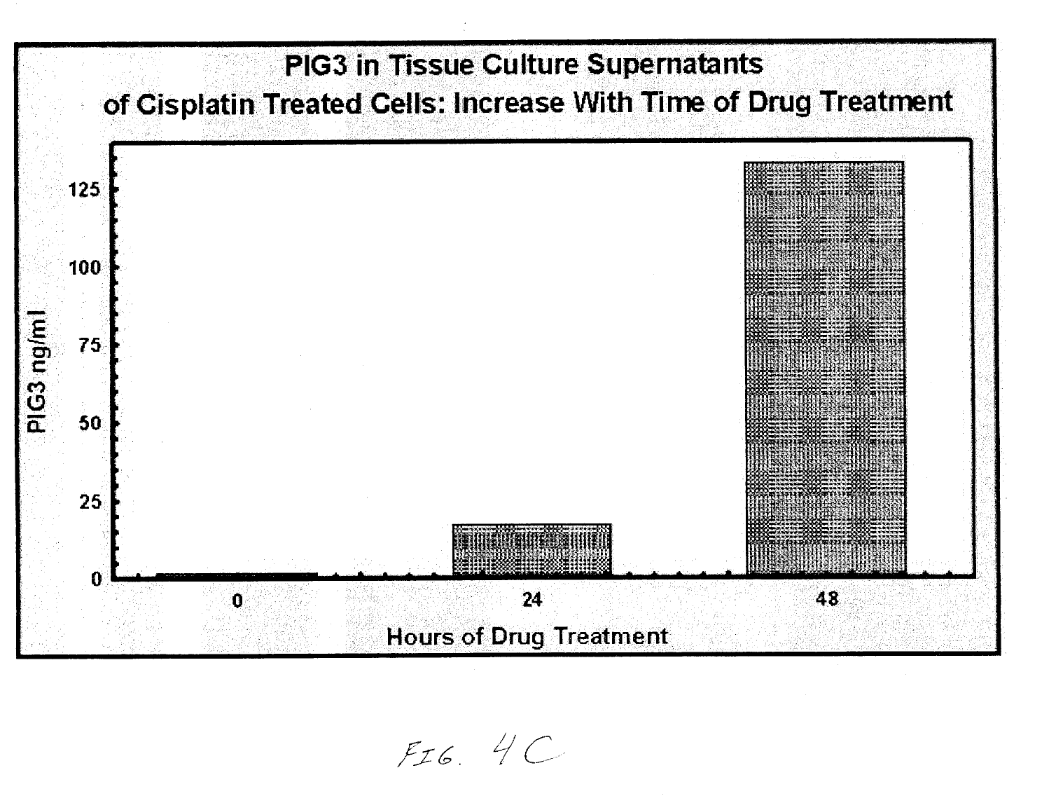

Also disclosed are methods for inducing the release of PIG3 in soluble form from cells. Additionally, the measurement of PIG3 along with another marker of DNA damage induced during patient treatment with DNA damaging agents such as cisplatin (or other similar chemotherapy agents) like DNA adduct development is taught. The combination of the two measurements provides further insight into the dosing, tolerance and treatment efficacy in patients. Additional utility is derived from using PIG3 measurements for monitoring combination chemotherapies where at least one agent used is a PIG3 induction agent from the list: cisplatin (or all platin family members of agents), taxol, 5-FU, adriamycin, irradiation or any p53 inducible gene 3 inducer and the second is any other chemotherapeutic agent or process used in conjunction with cisplatin (or all platin family members of agents), taxol, 5-FU, adriamycin, irradiation or any p53 inducible gene 3 inducer. (FIGS. 4A-4C)

Those skilled in the art will recognize that any other p53 inducible gene besides PIG3 may be substituted for PIG3 in this system.

This invention relates generally to the use of body fluid-soluble PIG3 as cellular markers. More particularly, the invention relates to a method of monitoring the degree of p53 induced cell death in a tissue by monitoring the level of PIG3 and/or fragments thereof released from cells in a body fluid-soluble form.

The product of p53-inducible gene 3 (PIG3) is a cytoplasmic protein, which is induced by exposure to genotoxic agents before the onset of apoptosis during p53-mediated growth arrest. The induction of PIG3 is p53-dependent and occurs with delayed kinetics as compared with other p53 downstream targets, such as p21WAF1 and MDM2. When cell lines are treated with adriamycin, a DNA-damaging and apoptotic-inducing agent known to increase endogenous p53 levels, PIG3, like p21WAF1, is strongly induced in the cell lines with wild-type p53 genes, but not in the cell with mutant p53. When p53-mediated growth arrest is reversed, elevated levels of PIG3 are maintained even in cells that resumed cycling in the absence of ectopic p53 expression, demonstrating that PIG3 is a long-lived reporter for transient activation of p53.

The proline-rich region of p53 is required for PIG3 activation. Although p53 protein lacking this region (p53delta62-91) can still induce many p53-responsive genes, it will not induce PIG3. Such a p53 mutant induces growth arrest but not apoptosis. Some tumor-derived p53-mutants, especially M2461, retain the ability to activate transcription of MDM2, but specifically fail to induce the PIG3 promoter, thus resembling p53delta62-91. Further, p53delta62-91 and p53M2461 are defective for induction of apoptosis. PIG3 shares significant homology to TED2, a plant NADPH oxidoreductase. TED2 is one of the few genes implicated in the apoptotic process necessary for the formation of plant meristems. The closest relative of PIG3 in mammals is an NADPH-quinone oxidoreductase that is a potent generator of Reactive Oxygen Species (ROS). ROS are powerful inducers of apoptosis. Expression profiles (SAGE-based) of p53-induced genes induce apoptosis by stimulating the production of ROS. Wild-type p53 is a tumor suppressor gene, which can activate or repress transcription, as well as induce apoptosis. The proteins encoded by p53 activated genes then collectively increase the content of ROS, which in turn damage mitochondria. Leakage of calcium and proteinaceous components from damaged mitochondria then stimulates the caspases that are activated during the apoptotic process.

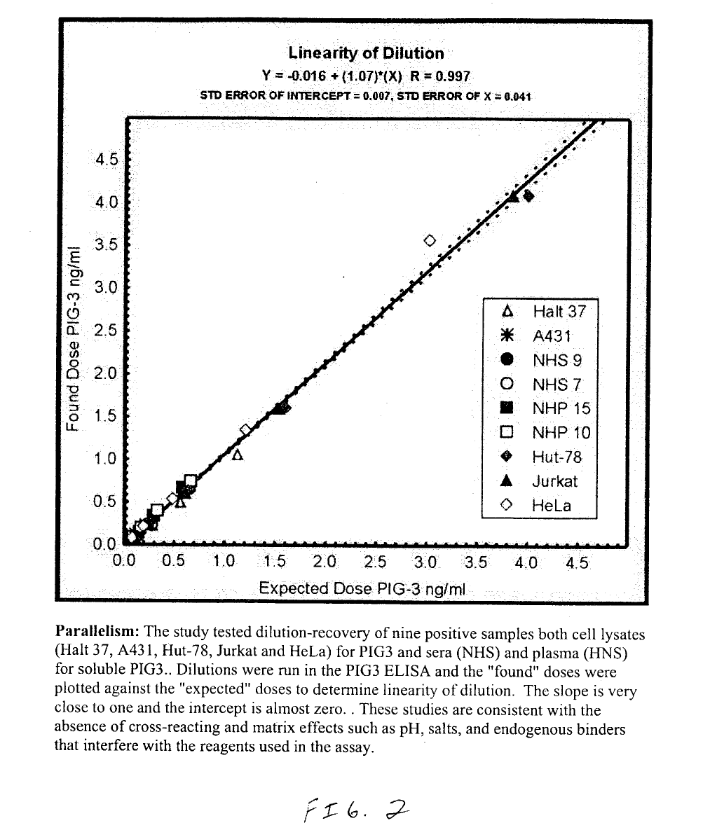

The invention results from the discovery that the product of p53-inducible gene 3 (PIG3) is present in human sera and plasma and in the tissue culture media of human cell cultures as well as in cell lysates (FIG. 2). Thus, the invention includes a method of determining the presence of PIG3 by assaying a fluid sample or cell sample obtained from the human patients for the presence of PIG3. The presence of PIG3 is a positive indicator of p53 activation and the presence of PIG3 in extracellular fluid is an indicator of apoptosis. The measurement of the level of PIG3 in the sample is useful in the diagnosis, prognosis, and treatment of DNA damage. Antibodies reactive with the proteins are disclosed as diagnostic and clinical research tools, and as a means for monitoring to determine the correct dosing of chemotherapeutic agents as in the immunoassay for quantitating the serum PIG3 proteins. Methods for preparing and purifying the proteins are also taught. The discovery was made possible by the development of an easy and quick immunoassay that detects PIG3 protein in human serum and plasma.

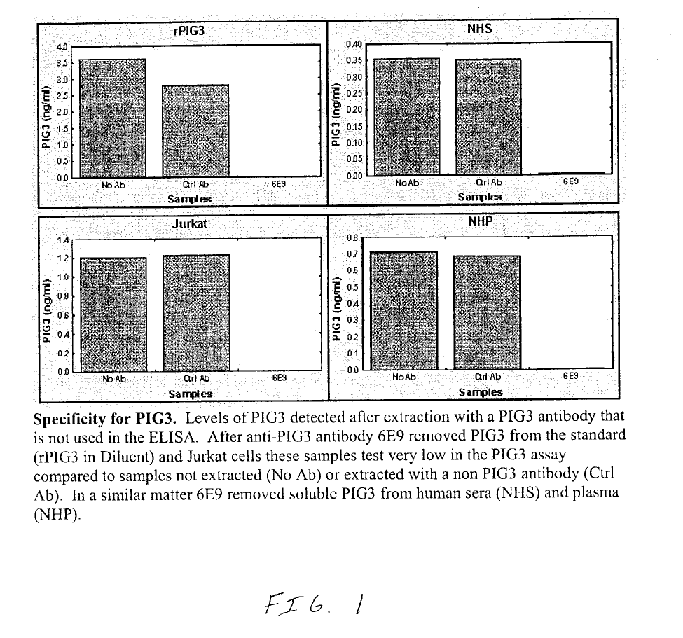

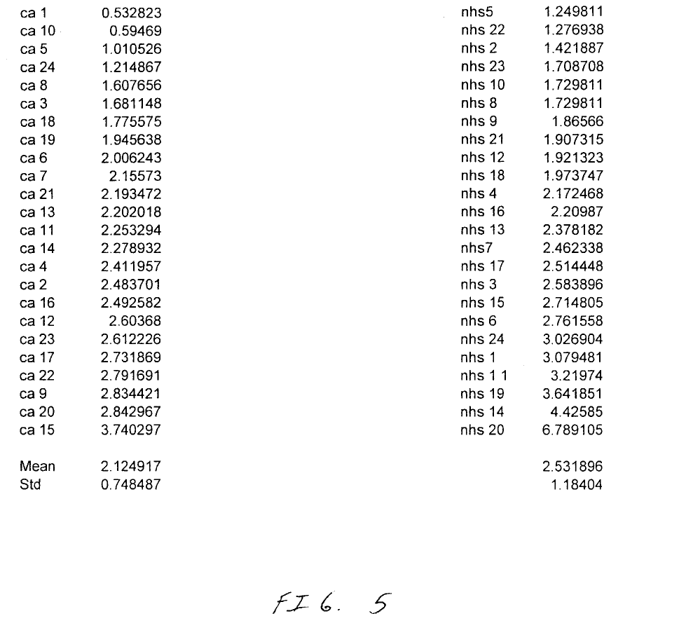

The PIG3 ELISA detects PIG3 protein in human sera and plasma samples and cell culture supernatants. FIG. 5, for example, shows drug induced in vitro PIG3 levels for cancer patient sera and normal human sera. Specificity was demonstrated by immunoaffinity extraction (inhibition of assay signal) of PIG3 positive samples by a specific PIG3 antibody (FIG. 1). The PIG3 antibody, which is not a component of the ELISA, extracted the PIG3 which leads to loss of signal in the assay (almost all the signal was lost), while the control antibody (any non PIG3) did not affect the signal of the PIG3 positive samples (FIG. 1).

Those skilled in the art will be able to recognize, or be able to ascertain, many equivalents to the specific embodiments of the invention described herein. All such equivalents not specifically mentioned as preferred embodiments are covered by the assays and methods of the invention as herein described and claimed, as are other combinations of techniques herein referenced and described, as may constructed or modified to provide similar methods.

Although specific features of the invention are shown in some drawings and not others, this is for convenience only as some feature may be combined with any or all of the other features in accordance with the invention.

Other embodiments will occur to those skilled in the art and are within the following claims:

1. A method for detecting the presence of PIG3 in a specimen, comprising the steps of:
(a) contacting a specimen with a solid support having immobilized thereon a first binding partner that binds specifically to a first binding site on a specimen-soluble PIG3 under conditions that permit said PIG3, if present in said specimen, to bind specifically to said first binding partner;
(b) contacting said solid support produced in step (a) with a second binding partner that binds specifically to a second binding site on said PIG3 under conditions that permit said second binding partner to bind specifically to said PIG3 thereby to produce an immobilized first binding partner-PIG3-second binding partner complex;
(c) contacting said complex with a third binding partner labeled with a detectable moiety, wherein said third binding partner binds specifically to said second binding partner, under conditions that permit said third binding partner to bind specifically to said second binding partner of said complex;
(d) measuring the amount of detectable moiety immobilized to said solid support, wherein the amount of immobilized detectable moiety is indicative of the quantity per unit volume of said pig3 in said sample, thereby to determine the quantity per unit volume of said pig3 in said specimen; and
(e) comparing the quantity per unit volume of said pig3 with a preselected value indicative of the presence of said degree of p53 activation and induction of cell death, wherein a quantity of said pig3 in said sample greater than said preselected value is indicative of the presence of said degree of p53 activation and induction of cell death in said specimen.

2. The method of claim 1, wherein said specimen comprises one or more body fluids selected from a group consisting of, blood, serum, plasma, urine, semen, spinal fluid, ascitic fluid, peritoneal fluid, saliva, sputum, and breast exudate.

3. The method of claim 1, wherein said body fluid comprises urine.

4. The method of claim 1, wherein said body fluid comprises serum.

5. The method of claim 1, wherein said degree of p53 activation and induction of cell death is caused by a disease condition, treatment of a disease, or progress of a disease.

6. The method of claim 5, wherein said disease is cancer.

7. The method of claim 6, wherein said cancer is carcinoma, adenoma, sarcoma, lymphoma or myeloma).

8. The method of claim 6, wherein said cancer is a bladder cancer, colon cancer, lung cancer, ovarian cancer, breast cancer, cervical cancer, kidney cancer, liver cancer, or uterine cancer.

9. The method of claim 6 wherein said cancer is a bladder cancer.

10. The method of claim 6, wherein said cancer is a colon cancer.

11. The method of claim 1, wherein said first binding partner is an antibody.

12. The method of claim 1, wherein said second binding partner is an antibody.

13. The method of claim 12, wherein said antibody is biotinylated.

14. The method of claim 1, wherein said third binding partner is streptavidin.

15. The method of claim 1, wherein the measurement of PIG3 is indicative of the efficiency of cell killing by radiation or chemotherapy.

16. A method for determining the optimum dosimetry of a therapeutic agent for a patient, comprising the steps of:
administering a first dose of said therapeutic agent to said patient;
measuring one or more quantity per unit volume of PIG3 in one or more specimens taken from said patient;
comparing said quantity per unit volume of said PIG3 with a preselected value that is indicative of cell death induction; and
administering a second dose of said therapeutic agent to said patient based on said step of comparing said quantity per unit volume of said PIG3 with said preselected value that is indicative of cell death induction.

17. A test kit, useful for performing an immunoassay to detect the presence of PIG3 in a specimen, comprising one or more monoclonal antibodies specific for the human PIG3 protein.

18. The test kit of claim 17, wherein one or more of said monoclonal antibodies is monoclonal antibody X1155P.

19. The test kit of claim 17, wherein one or more of said monoclonal antibodies is monoclonal antibody X1156M.

20. The test kit of claim 17, wherein one or more of said monoclonal antibodies is a biotinylated detector monoclonal antibody.

21. The test kit of claim 17, further comprising one or more immunologically responsive substances selected from a group consisting of horseradish peroxidase-conjugated streptavidin; radioactive agents, chemiluminescence agents, bioluminescence agents, fluorescence agents, or other chromophoric agents.

22. The test kit of claim 21, wherein one or more of said immunologically responsive substances comprises horseradish peroxidase-conjugated streptavidin to catalyze conversion of a chromogenic substrate tetra-methylbenzidine.

23. The test kit of claim 17, wherein one or more of said monoclonal antibodies is directly labeled.

24. The test kit of claim 17, wherein one or more of said monoclonal antibodies is indirectly labeled.
